(19) United States
(12) Patent Application Publication
(10) Pub. No.: US 2017/0137457 A1
(43) Pub. Date: May 18, 2017

(54) Labeled Nucleic Acids: A Surrogate for Nanopore-based Nucleic Acid Sequencing

(71) Applicant: Anastassia Kanavarioti, El Dorado Hills, CA (US)

(72) Inventor: Anastassia Kanavarioti, El Dorado Hills, CA (US)

(21) Appl. No.: 14/944,888

(22) Filed: Nov. 18, 2015

(51) Int. Cl. C07H 23/00; G01N 27/447; C12Q 1/68

(52) U.S. Cl. CPC C07H 23/00; C12Q 1/6869; G01N 27/44791

(57) ABSTRACT

Materials, methods, and systems for determining the sequence of a target nucleic acid are disclosed and described. Materials can include ssDNA, ssRNA, and dsDNA. Materials are first transformed to partially or fully osmylated single-stranded nucleic acid (osmylated or labeled polymer) after reaction with Osmium tetroxide 2,2′-bipyridine which labels selectively Thymidine over Cytidine, but leaves purines intact. Methods are provided to describe preparation of the osmylated polymers, their purification, and characterization. Labeled polymers are subject to voltage-driven translocation via nanopores of appropriate width so that the polymer can traverse as a single-file. The translocation is monitored and reported as a current vs. time (i-t) profile. The current is stable, but fluctuates during the polymer's translocation in a manner that pinpoints the osmylated bases interspersed among the intact bases. Methods are also described so that the events within the i-t profile unravel the sequence of the target nucleic acid.

### THIS APPLICATION CLAIMS THE BENEFIT OF U.S. PROVISIONAL APPLICATION

USPTO No. 62/083,256 filed on Nov. 23, 2014 entitled “Osmylated DNA, a superior material for DNA sequencing using nanopores”, by Dr. Anastassia Kanavarioti, inventor. The contents of the above are hereby incorporated by reference in its entirety into this application.

### GOVERNMENT SUPPORT

NIH grant via R01 GM093099 to Cynthia J. Burrows, Chemistry Department, University of Utah for supporting the work of Yun Ding (see 3. Below)

### PUBLICATIONS OF THE INVENTOR RELEVANT TO THIS INVENTION

1. Kanavarioti A, Greenman K L, Hamalainen M, Jain A, Johns A M, Melville C R, Kemmish K, and Andregg W. Capillary electrophoretic separation-based approach to determine the labeling kinetics of oligodeoxynucleotides, Electrophoresis 2012, 33, 3529-3543. PMID: 23147698
2. Kanavarioti A. Osmylated DNA, a novel concept for sequencing DNA using nanopores. Nanotechnology 2015, 26, 134003. PMID: 25760070
3. Ding, Y, Kanavarioti, A. “Single Pyrimidine Discrimination during Voltage-driven Translocation of Osmylated Oligodeoxynucleotides via the α-Hemolysin Nanopore”, submitted.
4. Kanavarioti, A. “A non-traditional Approach to Whole Genome ultra-fast, inexpensive Nanopore-based Nucleic Acid Sequencing”, Austin J Proteomics Bioinform & Genomics. 2015, 2(2), 1012.
5. Henley R Y, Vazquez-Pagan A G, Johnson M, Kanavarioti A, Wanunu M. “Osmium-Based Pyrimidine Contrast Tags For Enhanced Nanopore-Based DNA Base Discrimination”, PLoS One, 2015, 0142155.

### PUBLICATIONS AND PATENTS OF OTHERS IN THE SAME FIELDS OF SCIENCE AS THIS INVENTION

Palecek E. Probing DNA structure with Osmium Tetroxide Complexes in Vitro. Methods in Enzymology 1992, 212, 139-55. PMID: 1518446. Please note that under our conditions osmylation of the ribose is not detectable.
Maglia, G.; Heron, A. J.; Stoddart, D.; Japrung, D.; Bayley, H. Analysis of single nucleic acid molecules with protein nanopores. Methods Enzymol. 2010, 475, 591-623. PMID: 20627172
Wolna, A. H.; Fleming, A. M.; An, N.; He, L.; White, H. S. and Burrows, C. J. Electrical Current Signatures of DNA Base Modifications in Single Molecules Immobilized in the α-Hemolysin Ion Channel. Isr. J. Chem. 2013, 53, 417-430. PMID: 24052667
Mitchell, N.; Howorka, S. Chemical tags facilitate the sensing of individual DNA strands with nanopores. Angew. Chem. Int. Ed. Engl. 2008, 47, 5565-8. PMID: 18553329
Kumar, S.; Tao, C.; Chien, M.; Hellner, B.; Balijepalli, A.; Robertson, J. W. F.; Li, Z.; Russo, J. J.; Reiner, J. E.; Kasianowicz, J. J. and Ju, J. PEG-Labeled Nucleotides and Nanopore Detection for Single Molecule DNA Sequencing by Synthesis. Scientific Reports 2012, 2, 684.
Borsenberger, V.; Mitchell, N.; Howorka, S. Chemically labeled nucleotides and oligonucleotides encode DNA for sensing with nanopores. J. Am. Chem. Soc. 2009, 131, 7530-31.
Chang C H, Beer M, Marzilli L G. Osmium-labeled polynucleotides. The reaction of osmium tetroxide with deoxyribonucleic acid and synthetic polynucleotides in the presence of tertiary nitrogen donor ligands. Biochemistry. 1977, 16: 33-8.
Nomura, A., Okamoto, A. Reactivity of thymine doublet in single strand DNA with osmium reagent. Nucleic Acids Symp. Ser. 2008, 52, 433-4.

| Application # | Filed: | Issued: | Title |
|---|---|---|---|
| 20150152495 | Nov. 26, 2014 | Jun. 4, 2015 | Compositions and Methods for Polynucleotide |
| | | | Sequencing |
| WO2013/041878 | Sep. 21, 2012 | Mar. 28, 2013 | Analysis of a Polymer comprising Polymer Units |
| U.S. Pat. No. 5,795,782A1, | Mar. 17, 1995 | Aug. 18, 1998 | Characterization of individual polymer molecules |
| U.S. Pat. No. 6,015,714A1, | | | based on monomer-interface interactions |
| EP0815438B1, | | | |
| Claimed IP of | | | Use of solid state nanopores for detecting labeled |
| Oxford | | | ssDNA and dsDNA |
| Nanopore | | | |
| Technologies | | | |
| 7,825,248 | Jan. 23, 2009 | Nov. 2, 2010 | Synthetic nanopores for DNA sequencing |
| 20030099951, | Nov. 21, 2001 | May 29, 2003 | Methods and Devices for characterizing duplex |
| 6,936,433 | | | nucleic acid molecules |
| 20150119259 | Apr. 8, 2013 | Apr. 30, 2015 | Nucleic acid sequencing by nanopore detection of |
| | | | Tag molecules |
| 20150037788 | Oct. 17, 2014 | Feb. 5, 2015 | DNA sequencing by nanopore using modified |
| | | | nucleotides |
| 20130264207 | Dec. 16, 2011 | Oct. 10, | DNA sequencing by synthesis using modified |
| | | 2013 | nucleotides and nanopore detection |
| 20120142006 | Dec. 28, 2011 | Jun. 7, 2012 | Massive parallel method for decoding DNA and |
| | | | RNA |
| U.S. Pat. No. 9,005,425B2 | Sep. 7, 2011 | Apr. 14, 2015 | Detection of Nucleic acid Lesions and adducts using |
| | | | nanopores |
| U.S. Pat. No. 5,217,863A | Dec. 26, 1991 | Jun. 8, 1993 | Detection of mutations in nucleic acids |

### TERMS

As used herein, and unless stated otherwise, each of the following terms shall have the definition set forth below.

Osmylation—The reaction of a nucleic acid to form a nucleic acid conjugate where the T-bases are T(OsBp), or where all the pyrimidines are osmylated, (T+C)OsBp. Intermediate levels of T- and C-osmylation are possible, only that due to selectivity T is practically completely osmylated before C is osmylated.
Osmylated—material that was subject to osmylation

### A—Adenine;

### C—Cytosine;

DNA—Deoxyribonucleic acid; unless specifically mentioned all bases are deoxynucleotides.

### G—Guanine;

### T—Thymide

### U—Uracil

For the purposes of this document and the experiments described herein: T=dT, C=dC, A=dA, G=dG, U=dU, i.e. all the nucleotides here are deoxynucleotides. To identify the ribonucleotides the terms rA, rU, rC and rG will be used herein.

ss—single stranded
ds—double stranded
nt—nucleotide
bp—base pair
PBS—phosphate buffer saline
wt—wild type
α-HL or α-Hemolysin—the alpha Hemolysin nanopore

“Nucleic acid” or polynucleotide shall mean any nucleic acid molecule, including, without limitation, DNA, RNA and hybrids thereof. The nucleic acid bases that form nucleic acid molecules can be the bases A, C, G, T, U, in the deoxy or the ribodeoxyform, as well as derivatives thereof that comprise the so called non-canonical or rare bases found mostly in tRNAs.

OsBp or Osbipy—Osmium tetroxide 2,2′-bipyridine (see FIG. 1)
nanopore or channel—natural or solid-phase nanopores, channels, hybrids thereof, or massively parallel devices or instruments including them.
CE—Capillary Electrophoresis: Typical methods comprise an untreated fused-silica capillary (50 um ID×40 cm) with extended light path purchased from Agilent. Typical buffers were 50 mM phosphate pH 7 or 50 mM borate pH 9.2 with 25 kV or 30 kV. With platinators a 0.1N NaOH wash was added after each analysis, to improve capillary performance.
HPLC—High Performance Liquid Chromatography: Typical methods comprise Ion-exchange with DNA-PAC PA200 HPLC column and a salt gradient at neutral or basic pH.

### BACKGROUND OF THE INVENTION

### DNA Sequencing:

The rapid, reliable, and cost-effective analysis and sequencing of nucleic acids is a major goal of government, researchers, and medical practitioners. The ability to determine the sequence of the bases in DNA has additional importance in identifying genetic mutations and polymorphisms. Established DNA sequencing technologies have considerably improved in the past decade, but still require substantial amounts of DNA and several lengthy steps, while struggling to yield contiguous read-lengths of greater than 500 nucleotides. This information must then be assembled “shotgun” style, an effort that depends non-linearly on the size of the genome and on the length of the fragments from which the full genome is constructed. These steps are expensive and time-consuming, especially when sequencing mammalian genomes.

The present invention combines, for the first time, two separate fields of chemistry into one system that can sequence a target nucleic acid with no limit in length, inexpensively, 100 to 1000-times faster than currently done, and more accurately. Typical processes, accompanying sequencing, of assembly and scaffolding that result in sequence ambiguities are also avoided. The first field involves nanopores as single molecule analytical devices, and the second field involves labeled nucleic acids, including osmylated nucleic acids.

Nanopore-based sequencing has been investigated for the last 20 years as an alternative to traditional sequencing approaches. This method involves passing a nucleic acid, for example single stranded DNA (ssDNA), through a nanometer wide opening while monitoring a signal, such as an electrical signal, that is influenced by the physical properties of the nucleic acid subunits as the analyte passes through the nanopore opening. The nanopore optimally has, at least one section, of the appropriate size and the three-dimensional configuration that allows the analyte to pass in a sequential, single file order. Under theoretically optimal conditions, the polymer molecule passes through the nanopore at a rate such that the passage of each discreet subunit of the polymer can be correlated with the monitored signal. Differences in the chemical and physical properties of the subunits that make up the polymer, for example, the nucleotides that compose the ssDNA, result in characteristic electrical signals. Nanopores, such as for example, protein nanopores held within lipid bilayer membranes and solid-state nanopores, which have been used for analysis of DNA and RNA, provide the potential advantage of robust analysis of polymers even at low copy number.

However challenges remain for the full realization of such benefits. For example, the five nucleotides (A, G, T, C, U) that are the canonical subunits of nucleic acids are chemically comparable and produce similar signals during translocation, therefore making their discrimination challenging. Additionally, nanopores are entities of definite length, and have recognition sites for a sequence of nucleobases, in contrast to recognition for a single base. Hence the observed signal corresponds to a sequence and not a single base, making the correlation of the signal to a single base questionable. All these issues create unacceptable error in “base-calling”. Another major issue with nucleic acid translocation via nanopores is that translocation per base is too fast to be resolved by contemporary state-of-the-art instruments. In order to address this problem, the field has instituted the use of enzymes, polymerases and others, which have the ability to move the nucleic acid one base at a time.

This development has been used with relative success, slowing down the translocation to easily detectable levels. Nevertheless such enzymes have proofreading functions and they do not always move the strand forward. Moreover the enzyme's movement is sometimes interrupted, which confuses the reading process, i.e. some parts of the nucleic acid are either read twice or not at all. Furthermore these enzymes are costly, and relatively slow in processing the strand. Specifically the enzymatic assistance results in translocation speeds that are 100 to 1000-fold slower compared to what current state-of-the art instruments can detect. The additional drawback of the enzymes is that they typically dissociate from the nucleic acid and sequencing is interrupted, yielding typical reading lengths that are less than 5000 nt. Hence the development of a sequencing technology that avoids enzymatic assistance is urgently needed.

Accordingly, a need remains to avoid the use of enzymes, a need to find another way to slow down the translocation of nucleic acids via nanopores, and also a need to clearly distinguish each nucleobase from the others. The methods and compositions of the present disclosure address all three issues, and related needs of the art.

### Nucleic Acid Labeling Agents:

In the 1960s nucleic acids were reacted with metalorganic labels, used as contrast agents, and evaluated as substrates for obtaining sequencing information by electron microscopy. Osmium tetroxide 2,2′-bipyridine (OsBp) was exploited as an agent to label the pyrimidines in both ssDNA and ssRNA, and monofunctional platinators were exploited as agents to label the purines. Frequently OsBp was also used to label unpaired Ts in dsDNA, followed by cyclic voltametry detection. Cis-platin is a bifunctional platinator, known to react with adjacent Gs, but it has additional reactivity and forms crosslinks between strands, so it is not a useful label for sequencing purposes. The EM sequencing approach encountered a number of obstacles and did not yield tangible results.

Among the unresolved issues that prohibits investigators from pursuing the labeling nucleic acids approach are (i) efficient and homogeneous labeling has not been reported, and (ii) no validated analytical tool exists to check a labeled polymer base by base and determine false positives and false negatives. Most importantly it is known that ss nucleic acids have tertiary structure, and hence the conjecture is made that the tertiary structure prohibits homogeneous labeling. Homogeneous labeling is a critical attribute for any nucleic acid label intended to facilitate sequencing. If labeling does not occur homogeneously, i.e., independent of length, sequence and composition, then the number of false negatives would be large and unpredictable, leading to erroneous “base calling”.

In this invention we describe methods that yield predictable and homogeneous labeling independent of nucleic acid length, sequence, composition, and tertiary structure, as well as analytical methods to determine and confirm the extent of labeling. We disclose and describe specific protocols that osmylate any nucleic acid to exactly the same extent, i.e., % T(OsBp) and % C(OsBp) without prior knowledge of sequence, length or composition even when this polymer has tertiary structure. The specific and substantial utility to label any unknown nucleic acid in a predictable way can be implemented to yield sequence information of the unknown nucleic acid as will be described in the “Detailed description of the invention” section.

### BRIEF SUMMARY OF THE INVENTION

This invention combines two different fields of chemistry, nanopores and osmylated nucleic acids, in a novel way that is utilized for fast, accurate, and inexpensive nucleic acid sequencing. We claim invention relating to the methods to label nucleic acids predictably, purify, and analyze the labeled polymer in order to confirm extent of labeling. We also claim invention as it pertains to utilizing osmylated nucleic acids via nanopore measurement that may yield sequencing of the target strand.

In 2012 the present inventor, as the leading scientist, published a physicochemical study on labeling oligos with OsBp, to show that, by using a recommended protocol, T-osmylation in oligos up to 80-mer is independent of composition, sequence, and length (Part A). There is no obvious connection for the results of that study with this invention. However in 2014 the present inventor submitted the above provisional patent and published in 2015 a study showing that, in addition to T-osmylation, C-osmylation in oligos is also independent of sequence, composition, and length. Furthermore by including a 7456 nt long circular DNA together with the oligos, it was shown that the independence carried on to long DNA (Part B). Based on this later study the labeling was now presenting a novel and non-obvious way of sequencing DNA by using a characterized surrogate, i.e. the osmylated material of the target DNA.

Experiments proposed by the inventor and conducted by collaborators at the University of Utah, using labeled polymers prepared and sent by the inventor, showed clear proof-of-concept using α-HL as the nanopore. Comparable experiments at another collaborator at Northeastern University in Boston using solid-state nanopores also confirmed the utility of osmylated DNA. Therefore the postulate of “nanopore-based sequencing using osmylated DNA as a surrogate”, has been validated in two different nanopore platforms, and osmylated DNA, using the methods disclosed in this invention presents a novel and substantial utility in the genome sequencing field (Part C). In the section on “Detailed description of the invention” we include all the evidence (Parts A, B, and C) that led to this invention.

In some embodiments the pyrimidine-specific label is osmium tetroxide 2,2′-bipyridine (OsBp). In some embodiments the nucleic acid is a short oligodeoxynucleotide (oligo) and the label is OsBp.

In some embodiments the nucleic acid is a long oligo (80-mer) and the label is OsBp.

In some embodiments the nucleic acid is a circular 7456-nt long DNA and the label is OsBp.

In some embodiments the labeled polymer is practically all T-osmylated, i.e., T(OsBp)-oligo or T(OsBp)-DNA.

In some embodiments the labeled polymer is completely (T+C)-osmylated, i.e., (T+C)(OsBp)-oligo or (T+C)(OsBp)-DNA.

In some embodiments the nanopore is wt a-Hemolysin (α-HL) and the oligo is 20 nt long with one dT(OsBp).

In some embodiments the nanopore is α-HL and the oligo is 20 nt long with one dC(OsBp).

In some embodiments the nanopore is α-HL and the oligo is 20 nt long with one 5′Me-dC(OsBp).

In some embodiments the nanopore is α-HL and the oligo is 20 nt long with one dU(OsBp).

In some embodiments the nanopore is α-HL and the oligo is 23 nt long with four units dT(OsBp) interspersed among intact nucleotides.

In some embodiments the nanopore is α-HL and the oligo is 23 nt long with four units dT(OsBp) and 5 units dC(OsBp) interspersed among intact purines.

In some embodiments the nanopore is α-HL and the oligo is 48 nt long with four units T(OsBp) interspersed among intact nucleotides.

In some embodiments the nanopore is α-HL and the oligo is 48 nt long with four units dT(OsBp) and 5 units dC(OsBp) interspersed among intact purines.

In some embodiments the nanopore is α-HL and the oligo is 80-mer with 24 units dT(OsBp) and 1-2 units dC(OsBp) interspersed among intact nucleotides.

In some embodiments the nanopore is α-HL and the oligo is 80-mer with 24 units dT(OsBp) and 17 units dC(OsBp) interspersed among intact purines.

In some embodiments the nanopore is solid-state (SiN) with 1.6 nm wide pore and the oligo is 80-mer with 24 units dT(OsBp) and 1-2 units dC(OsBp) interspersed among intact nucleotides.

In some embodiments the nanopore is solid-state (SiN) with 1.6 nm wide pore and the oligo is 80-mer with 24 units dT(OsBp) and 17 units dC(OsBp) interspersed among intact purines.

### DESCRIPTION OF DRAWINGS

Part A includes Tables 1 through 3 and FIGS. 2 through 11. Part B includes Tables 4, 5 and FIGS. 12 through 15. Part C includes FIGS. 16 through 23 and Tables 6 and 7. FIG. 1 is the label and is used in all the Parts.

FIG. 1 illustrates the reaction between Osmium tetroxide and 2,2′-bipyridine that forms a complex with a small equilibrium constant. This complex (bipy-OsO4, or Osbipy, or OsBp) reacts in a next step with a pyrimidine (deoxythymidine monophosphate shown) by addition to the C5-C6 double bond of the pyrimidine ring to form a conjugate. A similar product is formed by addition to the C5-C6 double bond of cytidine or uracil. The reaction is independent on the presence of ribose or deoxyribose as well as independent of whether the reactant is a nucleoside or a nucleotide, mono-, di-, or triphosphate, or a unit within a polymer. The actual conjugate is a topoisomer formed by addition from the top or the bottom of the pyrimidine ring. Hence the products are two isomers that are resolved by capillary electrophoresis (CE) or High performance liquid chromatography (HPLC), as seen in later figures. In principle, the OsBp moiety does not interfere or prohibit base pairing. One way to illustrate the difference between osmylated and intact bases is to compare (molecular weight) of each: dC (111), dT (126), dA (135), dG (151); dC-OsBp (521), dT-OsBp (536), i.e. osmylation adds about 400% mass to the reactive base compared to an unreactive one. Instead of 2,2′-bipyridine, a X-substituted 2,2′-bipyridine at any one or more of the Carbon atoms replacing one or more Hydrogen can also be suitable for complexation with OSO₄, and exhibit comparable properties as OsBp.

TABLE 1 lists Oligos (ODN) used for the experiments illustrated in later Figures. Listed are the sequences, the SEQ ID NO (see Sequence Listing), # of T or C over total nucleobases (Nₜₒₜₐₗ), k_{obsd}, the rate of product formation with 3 mM Osbipy, and values for Infinity Ratio 320/260 for T-labeling or (T+C)-labeling; infinity ratio indicates the normalized absorbance once the specified reaction is practically complete.

FIG. 2 illustrates the reaction of OsBp with thymidine 5′triphosphate (dTTP). Specifically, it shows capillary electrophoresis (CE) profiles from a reaction of 2.2 mM Osbipy (migration time, mt, at 3 min, not shown) with 0.16 mM dTTP at 25° C. monitored at 260 nm; consecutive analyses of the same reaction mixture show that the dTTP peak decreases and a product peak, Osbipy-dTTP or (OsBp)dTTP, appearing as a doublet, increases with time. The formation of two peaks confirms the topoisomerism described in FIG. 1.

FIG. 3 illustrates the reaction of OsBp with an oligo. Specifically, it shows CE profiles from the consecutive analyses of a sample with 1.3 mM Osbipy and 0.08 mM oligoT1 (AAAATAAAA) in water at 25° C. Bottom profile shows the excellent stability (3 days) of the Osbipy-labeled-oligo (P1) in the presence of excess Osbipy at 25° C. P1 appears as two peaks because of the topoisomerism discussed in FIG. 1. At these concentrations the reaction does not go to full completion, as seen by the small peak remaining at the oligoT1 migration time (mt).

FIG. 4 illustrates the reaction of OsBp with an oligo. Specifically, it shows CE profiles from the consecutive CE analyses of a sample with 1.3 mM Osbipy and 0.08 mM oligoT2 (AAATTAAA) at 25° C. P1 stands for the mono-osmylated product (4 isomers expected, two may comigrate). P2 stands for the di-osmylated product. With these concentrations of starting materials and after 102 minutes the reaction has only produced about half of the final product, i.e. the di-osmylated oligoT2.

FIG. 5 illustrates the reaction of OsBp with an oligo. Specifically, it shows CE profiles from the consecutive analysis of a reaction mixture with 1.3 mM Osbipy and 0.04 mM oligoT3 (AATAATAATAA, SEQ ID No:1) at 25° C. Impurity present in the reaction mixture remains unchanged as shown. Reaction is practically completed overnight. Final product (P3, shaded column) exhibits excellent stability over 3 days at 25° C., even in the presence of excess label.

FIG. 6 compares the reactions of OsBp with two different oligos under the same conditions. Specifically, it shows CE profiles from the analyses of two samples incubated for 19 hours at 25° C.: Top, 1.3 mM Osbipy with 0.08 mM oligoT2 (see FIG. 4); bottom, 1.3 mM Osbipy with 0.08 mM oligoC2 (AAACCAAA). Impurity present in the oligoC2 reaction mixture remains unchanged during incubation. After 19 hours no oligoT2 can be detected, whereas oligoC2 shows very little reactivity. The appearance of multiple peaks for P1 and P2 has been discussed earlier.

FIG. 7 illustrates the rate of oligo disappearance as a function of the number of Ts (x-axis). The plot is shown for the oligos T1, T2, and T3 (see Table 1). Rates (y-axis in 1/min) increase proportionally with the number of Ts, as statistically expected when there is no interference or inhibition of the reaction of one T in the presence of additional Ts. Please note that in oligoT2, the two Ts are adjacent. The proportionality of the rate of disappearance of the substrate as a function of the number of Ts is a general observation that we have evidenced in many experiments. It is worth mentioning though that the rate of product appearance, i.e. the 100% osmylated product has a rate of formation that is independent of the number of Ts. For example, the rates of product formation for the above oligos are 0.050, 0.037, and 0.045 per min, respectively for oligoT1, T2 and T3. These rates are within experimental error the same. Basically any oligo becomes T-osmylated with the same rate independent of sequence, length, and composition (see 4^{th} column in Table 1).

FIG. 8 illustrates that under “best mode” conditions the concentration of the oligo, within constrains, does not affect the rate of product formation. Specifically, FIG. 8 shows data from the reaction of three different concentrations of oligoT8 (TTTTTTTT), at 0.025, 0.050 and 0.075 mM, with 3 mM OsBp at 25° C. Average value at the plateau is 1.51±0.02, and k_{obsd}=0.041 per min (rate of product formation) from all the data plotted together. Please note that the rate of complete osmylation of oligoT8 is, within experimental error, the same as for oligoT1, T2, and T3, in support of the underlined conclusion (see FIG. 7). The Ratio 320/260 stands for the ratio of the CE peak areas at 320 and 260 nm; it is a normalized measure for final product formation. R 320/260 equals zero at the onset of the reaction, because DNA does not absorb at 320 nm, but increases with time as more product is formed. The osmylated product has absorbance in the range of 320 nm, and the absorbance is proportional to number of osmylated units, normalized over the total number of nucleotides/units within the labeled polymer. More on the development of the UV-Vis assay that measures extent of osmylation is described in the “Detailed description of the invention” section.

FIG. 9 illustrates that consecutive 15 min CE analyses of reaction mixture composed of L1 (see Table 1; SEQ ID NO:5), an 80 nt long oligo, and 2.6 mM OsBp in water at 44° C. in the presence of 0.042 mM dCTP as internal standard. Analysis #1 is obtained after 3 min. OsBp migrates at 2.3 min (not shown) and it is a large peak compared to the others. Peak migration time (mt) of dCTP internal standard does not exhibit a shift. L1 peak mt shifts exponentially with time towards the peak mt attributed to L1-T(OsBp). In the absence of OsBp, L1 peak mt=6.4 min, approximately 0.4 min later compared to peak mt #1, clearly showing that even after 3 min there is substantial labeling.

FIG. 10 illustrates that migration time (mt) of main peak as a function of incubation time at 44° C. for the reaction of long oligo L1 with 2.6 mM Osbipy (see FIG. 9). The point at 0 time corresponds to the mt of the intact 80-mer (L1) analyzed under the above conditions.

FIG. 11 illustrates a linear correlation between the Ratio 320/260 for oligos that are practically 100% T-osmylated (Infinity Ratio 320/260) with the fraction of Ts over the total number of nucleobases (T/Nₜₒₜₐₗ). Data obtained from Table 1. The best fit of the data is linear with slope=1.53, and goes through the origin. This linear correlation is consistent with the proposition that each T-osmylated unit contributes the same chromophore.

TABLE 2 lists the selectivity values obtained for the reaction between OsBp with a mixture of dTTP+dCTP in competition experiments. Experimental details are included in the footnote of Table 2.

TABLE 3 lists extent of osmylation, separately % of T-osmylated and % C-osmylated for a random sequence oligo as a function of incubation time, or half-lives of the T-osmylation process. The values are calculated based on the pseudo first-order kinetics that are implemented in these studies. All experimental detail is included in the footnote of Table 3. For 60 minutes incubation under the specified conditions (2^{nd} preferred mode), each oligo will have 90% T-osmylated and 6.5% C-osmylated content, independently of sequence, composition, and length.

TABLE 4 lists Oligos/DNA, SEQ ID NO, sequences and purity, used in experiments illustrated in the following figures.

TABLE 5 lists the Oligos/DNA from Table 4 together with the number of Ts and Cs and the total number of nucleobases, Nₜₒₜₐₗ. R1 (312/272) and R2 (312/272) are given by the ratio of the peak area at the two different wavelengths following protocol A and protocol B, respectively. Protocols A and B (2^{nd} preferred mode) are described in the section for “Detailed description of the invention”. R1 and R2 (312/272) are optimized measures and replace the measure R (320/260); explanation is given in the “Detailed Description of the Invention”.

FIG. 12 illustrates CE profiles of sequential analyses monitoring the osmylation of Oligo10 (AAACACACACACACAA, with 6 Cs; SEQ ID NO:18) at 272 nm every 17 min. Mixture contains 300 ng/μL Oligo10 with 7.9 mM Osbipy at 27° C. in water. T1 is obtained after only 6 min from mixing the reactants. Still one can clearly detect two groups of peaks: Group C1 is believed to be the singly osmylated Oligo10. C1 has multiple peaks due to the six plausible positional isomers. Please note that CE is known to resolve positional isomers. Group of peaks, designated C2 (shaded block), represents products with two osmylated Cs, C3 with three osmylate Cs, C4 with four (shaded block), C5 with five and C6 with six osmylated Cs (shaded block). Even after many hours there was no additional peak migrating ahead of C6. However these conditions, i.e., relatively high concentration of oligo and only 7.9 mM osbipy do NOT lead to complete osmylation and the reaction levels off. The reason we chose to show these conditions is because the reaction is relatively slow compared to the CE analysis and one can clearly follow the appearance of higher osmylated products and the accumulation of the material initially from the Oligo to the C1, and then to C2, followed by accumulation to C3 (see CE profile T3) and to C4 (see CE profile T4, bottom). Please note that in T4 the Oligo peak is undetectable. Identification of products, as specified here, is supported by the observation that R(312/272) of a certain group peak is proportional to the proposed number of Cs. Separate and well resolved group of peaks, such as the ones observed with Oligo10, were not observed with either Oligo8 or Oligo9 (see Table 4), even though their composition is identical to Oligo10.

FIG. 13 illustrates overlapping profiles of three CE analyses of M13mp18 from Bayou Labs (for simplicity M13). Peak labeled M13: CE profile of the intact DNA monitored at 272 nm. The profile of the sample monitored at 312 nm is also included in the figure, but no peak is detectable due to the negligible absorbance of M13 at 312 nm. Peak labeled M13(R1): CE profile of the product of the reaction of M13 with Osbipy according to Protocol A, followed by TrimGen purification. Osbipy peak, if detectable, would appear at about 3.5 min. As seen by comparing the two traces under M13(R1), this material absorbs more at 272 nm compared to 312 nm. Peak labeled M13(R2): CE profile of the product of the reaction of M13 with Osbipy according to Protocol B, followed by TrimGen purification. In contrast to M13(R1), M13(R2) absorbs more at 312 nm compared to 272 nm (see Table 5). Please note that the concentrations of these three materials are not the same, and this is why their respective peak areas differ.

FIG. 14 illustrates the correlation between Ratio of the peak area at 312 nm vs 272 nm for the osmylated product peak following Protocol A, R1 (312/272), as a function of the fraction of thymidine bases over the total number of bases in an oligo/DNA, T/Nₜₒₜₐₗ. Line is forced via the intercept. Oligos/DNA used in this study and the data plotted here are listed in Table 5.

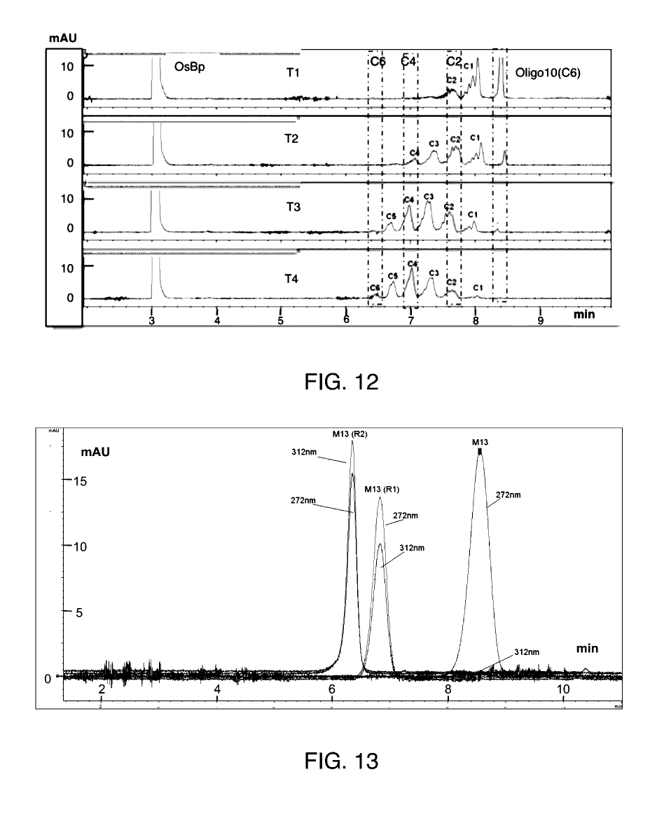

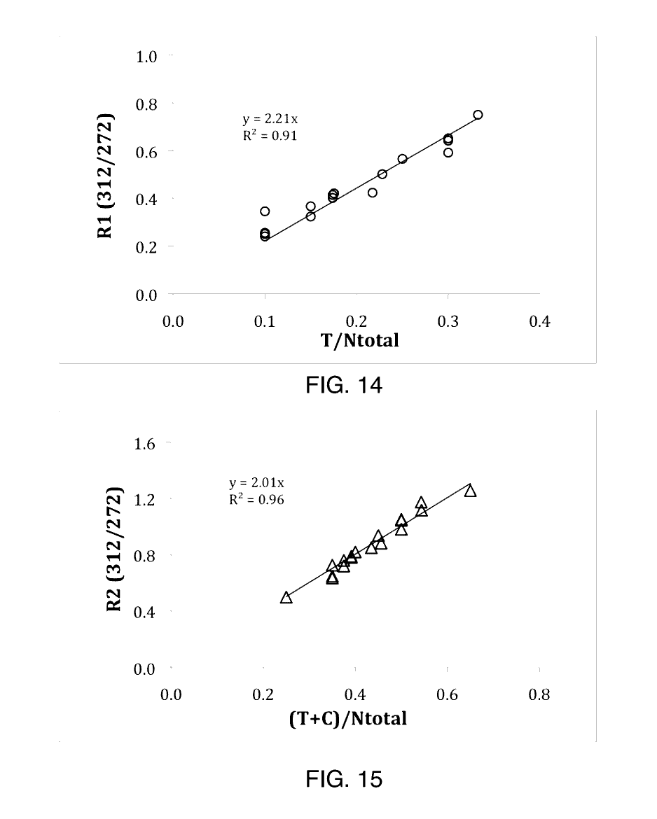

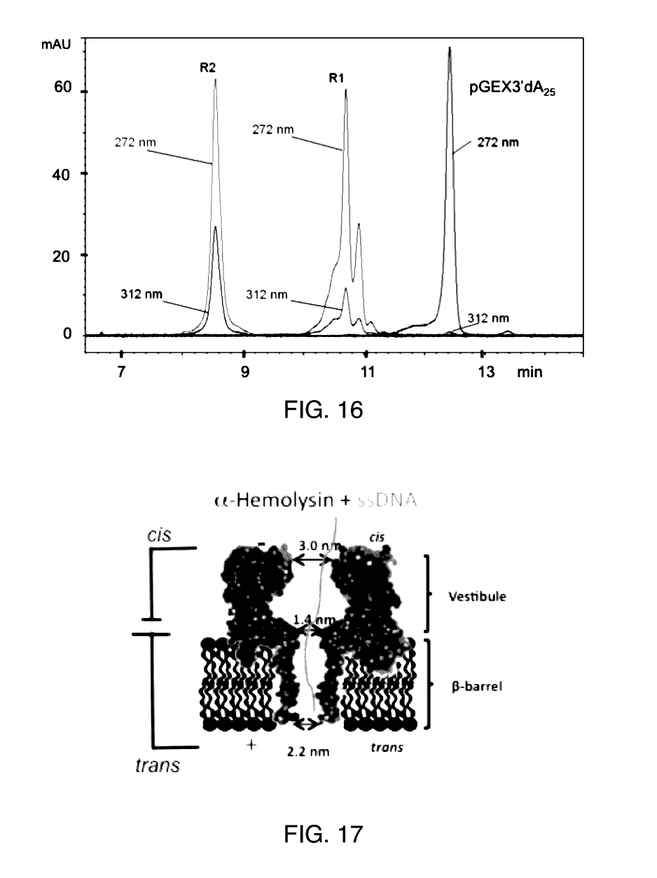

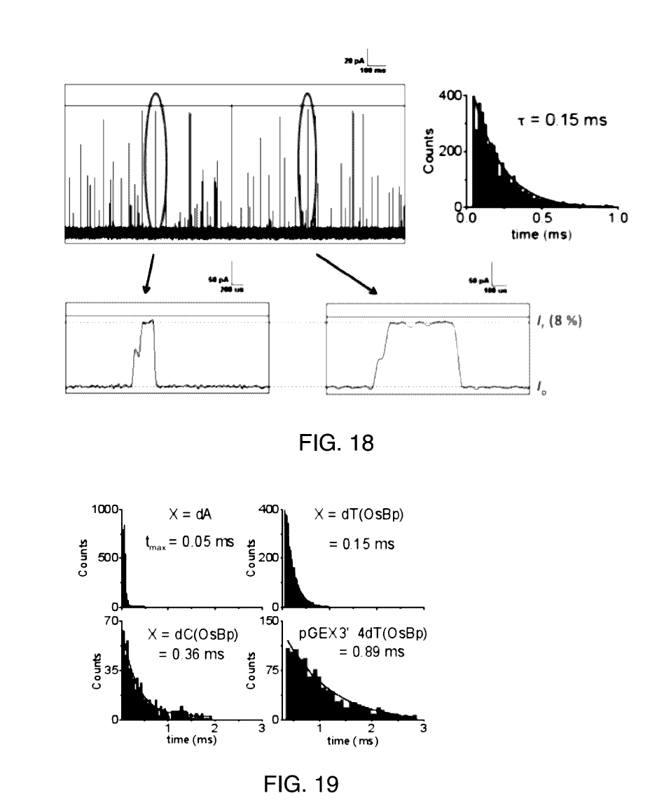

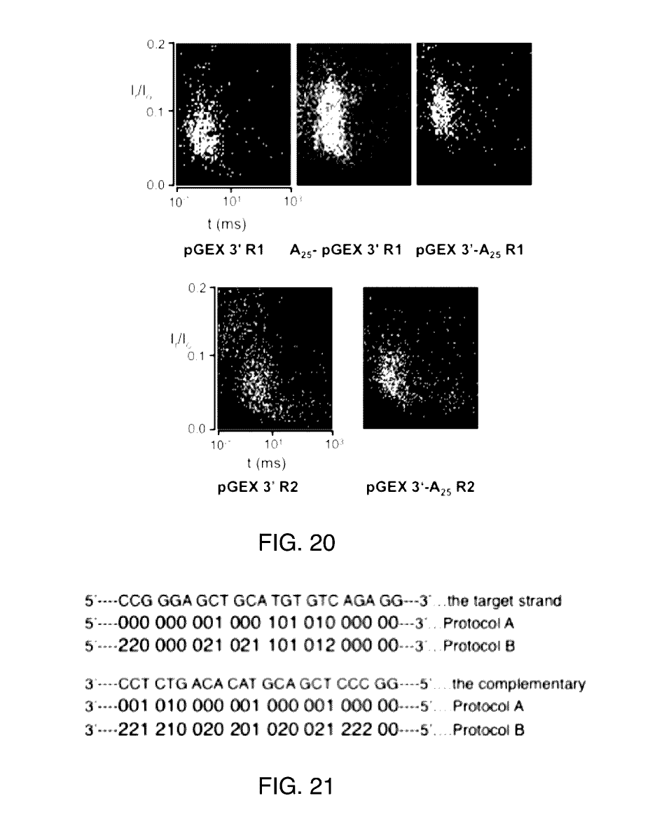

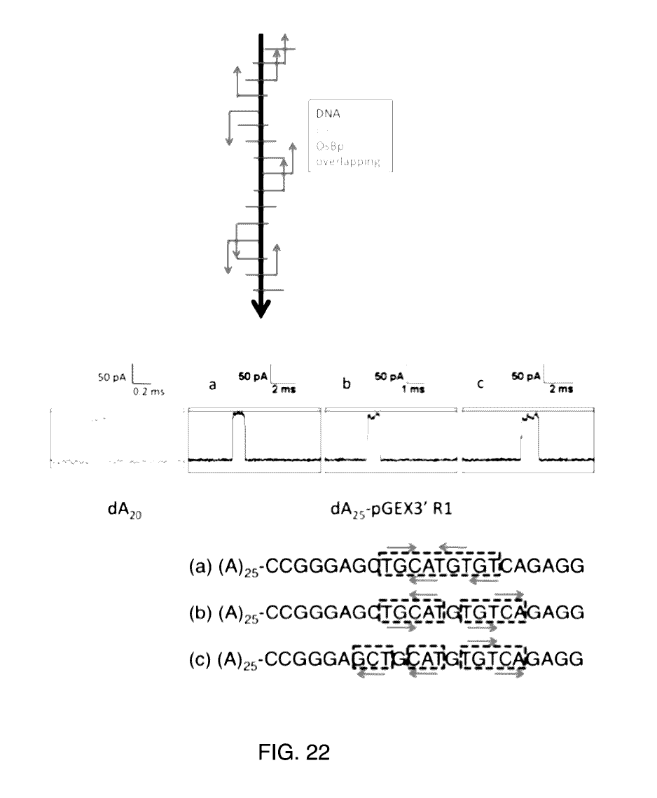

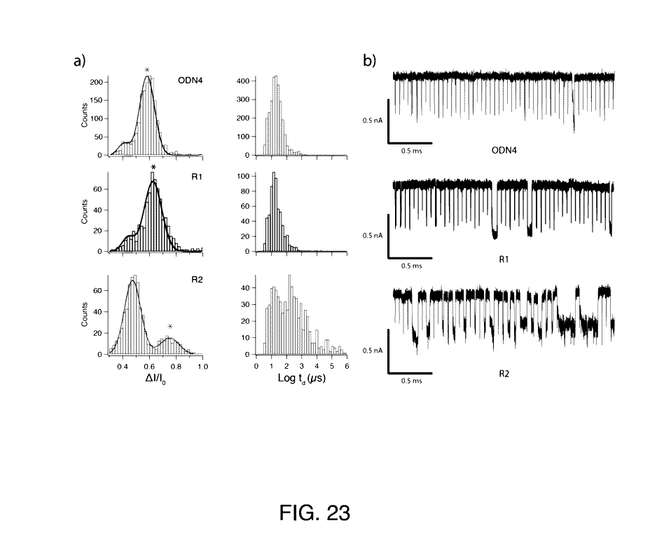

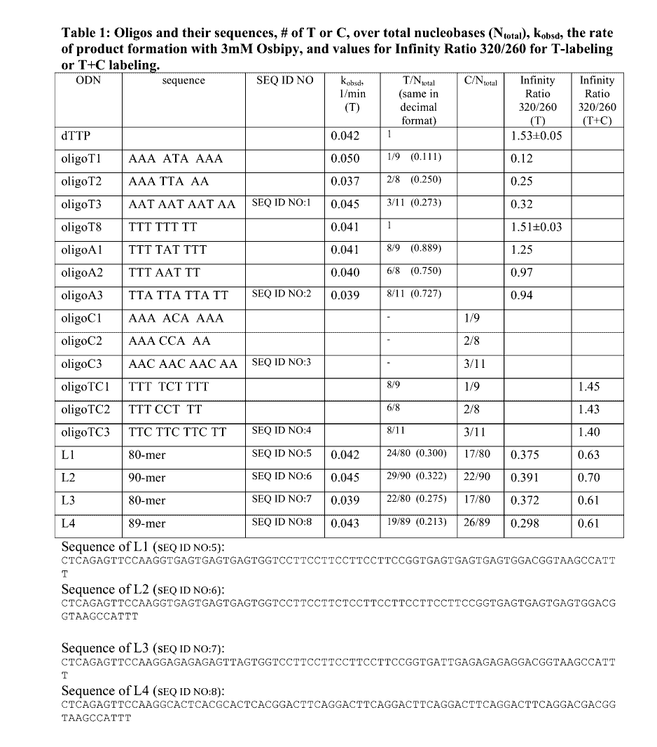

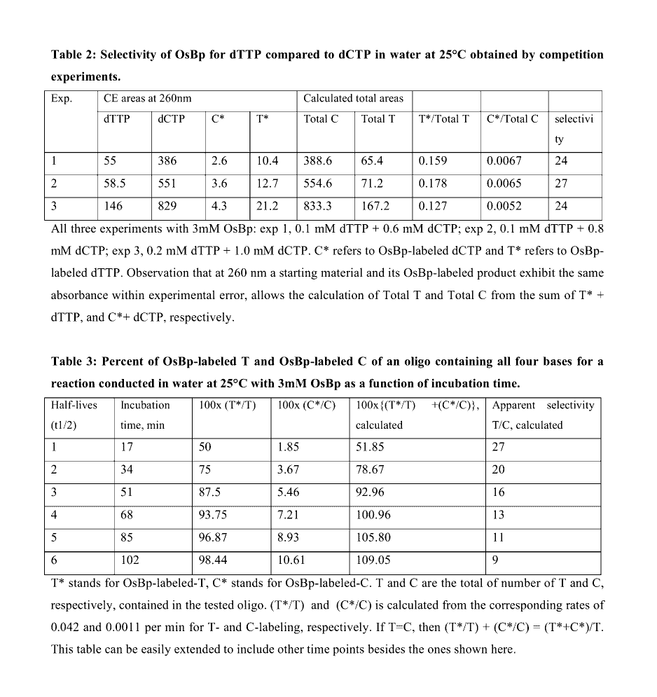

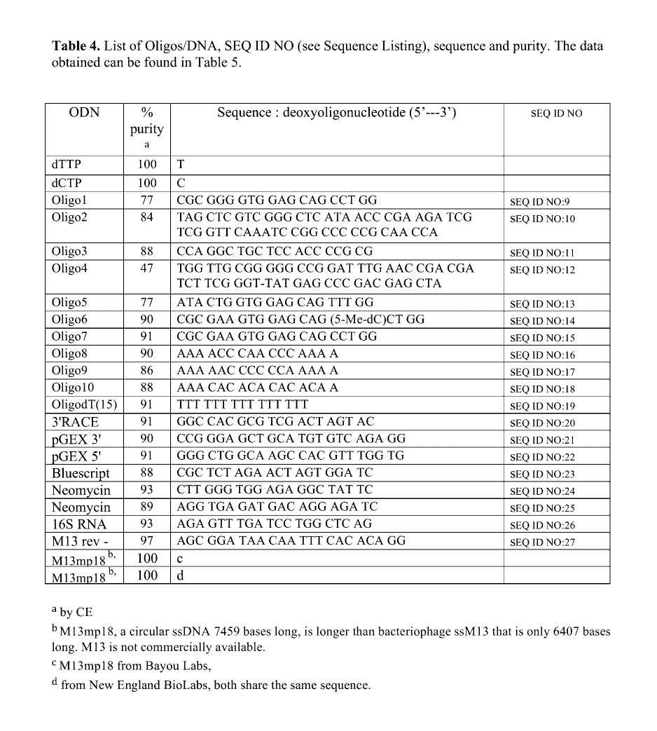

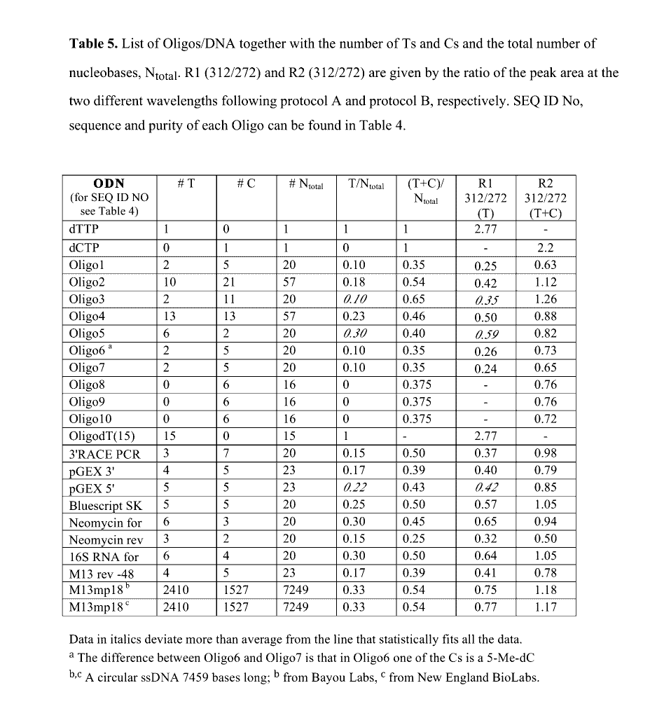

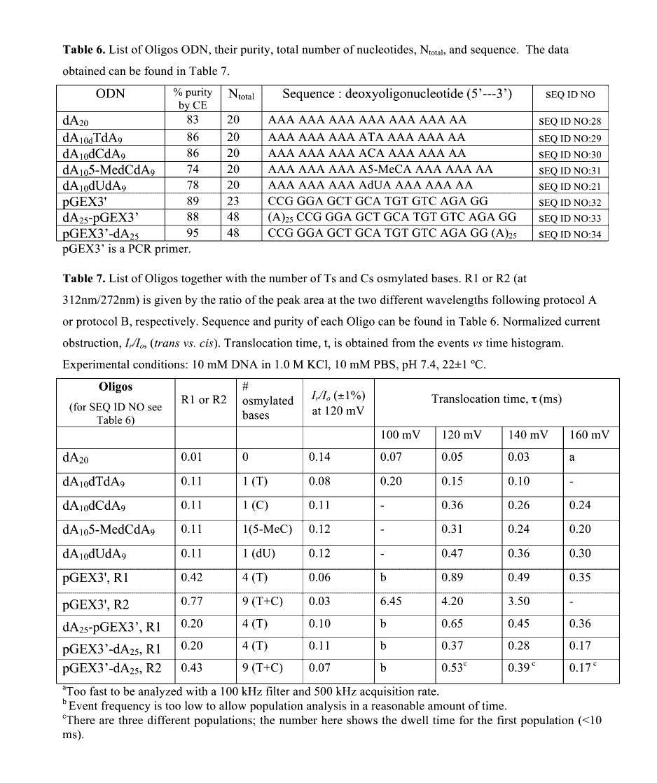

FIG. 15 illustrates the correlation between the Ratio of the peak area at 312 nm vs 272 nm for the osmylated product peak following Protocol B, R2 (312/272), as a function of the fraction of pyrimidines over the total number of bases in an oligo/DNA. Line is forced through the intercept. Oligos/DNA used in this study and the data plotted here are listed in Table 5. The R312/272 measure reflects an improvement we made on the assay to gain better sensitivity (earlier measure R320/260), as will be shown by comparing the slope of FIG. 14 with the one from FIG. 11, the later being of lesser value (2.21 vs. 1.53).

FIG. 16 illustrates CE overlapping traces of oligodeoxynucleotides pGEX3′-dA25 intact (SEQ ID NO:34) and pGEX3′-dA25 at R1 and R2 levels of osmylation per Protocols A and B, respectively (sequence in Table 6). Materials are at comparable, but not identical, concentrations. Migration is in the order of intact oligo last, R2 early, and R1 in the middle. Traces are shown at two wavelengths, at 272 nm and 312 nm, to illustrate that DNA exhibits about 1% absorbance, whereas R1 and R2 absorb substantially, and R2>R1. The detail in the R1 peak is attributed to different topoisomers produced from either top or bottom addition to the C5-C6 double bond. Topoisomers exist also with R2, but are too many to be resolved. The ratio R(312 nm/272 nm) is a normalization, and the wavelengths are selected to maximize the value of R.

FIG. 17 illustrates a representation of the translocation of ssDNA via the α-Hemolysin nanopore (α-HL) showing the 1.4 nm constriction zone and the rather long but confined b-barrel; voltage (positive, trans to cis) across the insulated nanopore leads to ion current via the pore and threading of the ssDNA, which obstructs the current when inside the pore.

FIG. 18: Top Left, Observed current vs time (i-t, in pA vs. ms) profile shown for dA₁₀dT(OsBp)dA₉ (SEQ ID NO:29) via the α-HL nanopore at 120 mV in 1M KCl, pH 7.4 with 10 mM PBS, at 22° C. (see Tables 6 and 7 below). Top Right, dwell time for all the translocation events with comparable (in the range 85 to 95%) current obstruction. Average dwell time t=τ=0.15 ms. Bottom: Two events selected and magnified (time in μs) to show the current obstruction at a relative residual current of 8%. Events with relatively low residual current (lower than 80%) are attributed to events other than complete translocation of the DNA.

FIG. 19: Summary illustration of the counts vs. time obtained from the nanopore experiments with α-HL, as described in FIG. 18. Exponential treatment of the data provides the dwell times for four different oligos. Sequence of the first three is dA10XdA9, and X is identified in the figure (SEQ ID NO: 28, 29, 30). Sequence of pGEX3′ (SEQ ID NO:21) is listed in Table 5. Table 6 incorporates the data. Notably osmylation of even one unit in an oligo has a marked slowing down effect in the translocation of the oligo, a feature that has never been observed before in the nanopore field. There is a dramatic difference in the translocation properties between the three 20-mers that differ only in one nucleotide. Even more surprising is the observation that osmylated-T is sensed dramatically different from osmylated-C with dwell times at 120 mV t=0.15 ms vs t=0.36 ms, respectively. This large discrimination enables the nanopore-based, enzyme-free, labeled DNA sequencing claimed in this invention (see Sequencing strategy in FIG. 21 and in the “Detailed description of the Invention” section).

Table 6: List of oligos with SEQ ID NOs, and their sequences used in the α-HL translocation experiments. The osmylation products CE profiles of the last entry can be found in FIG. 16.

Table 7: Translocation parameters, i.e. residual current and dwell time, reported for four different conditions, 100, 120, 140 and 160 mV. Representative data at 120 mV are illustrated in FIG. 19.

FIG. 20: Plots of relative residual current (Iᵣ/Iₒ) as a function of time shown as intensity plots or heat plots. Comparison of oligos with SEQ ID NO:21, 33 and 34. These plots illustrate that the A₂₅ tail facilitates translocation by reducing the current obstruction and that the tail at the 3′-end is more facilitating compared to the tail at the 5′-end. The advantage of the A₂₅ tail is seen both with T-osmylation only (top figures) as well as with both (T+C)-osmylation (bottom figures). The effects observed here regarding the A₂₅ tail are in agreement with literature precedent for intact oligo translocation.

FIG. 21: Sequencing strategy where 1=dT(OsBp) and 2=dC(OsBp). Oligo shown, as example, is pGEX3′ (SEQ ID NO:21). Since α-HL nanopore discriminates between dA, dT(OsBp) and dC(OsBp), with dwell times of τ=0.05, 015 and 0.37 ms, respectively, then sequencing is possible by “reading” the i-t signals. Based on literature no discrimination is expected for dA vs dG. Hence for successful sequencing both the target strand and its complementary should be sequenced. Sequencing of the complementary strand is necessary, so that the A and G in the target strand can be identified via the corresponding T and C in the complementary. (i) Protocol A yields 90% dT(OsBp) and 6.5% dC(OsBp); Protocol B yields practically 100% osmylated pyrimidines, both dT(OsBp) and dC(OsBp). As shown experimentally (see Table 7) α-HL discriminates also by relative current levels between dA, dT(OsBp), and dC(OsBp), even though this discrimination by relative current is not nearly as dramatic as the dwell time.

FIG. 22 (Top): Osmylated DNA strand representation to show the approximately parallel line up of OsBp moieties along the strand, the top or bottom conjugation of OsBp with the nucleobase, the extension of OsBp to obscure next-door neighbor, and the plausible overlap of two OsBp moieties. The later is consistent with the observed much slower translocation time for oligos with multiple osmylated pyrimidines (see Table 7, PGEX3′ R1 and R2 (SEQ ID NO:21)). In this two-dimensional representation some interactions appear artificially close and others apart. Please note that in ssDNA, adjacent bases can take positions practically across from each other in order to minimize next-door neighbor OsBp interactions. (Middle): Sample i-t traces for the control dA₂₀ (SEQ ID NO:28) and for dA₂₅-pGEX3′ (SEQ ID NO:33) R1 (with 4T(OsBp)) are attributed to Bottom: (a) continuous blockage, (b) blockage interrupted once, or (c) blockage interrupted twice; “interruptions are attributed to the passing of an intact base (dG) in FIG. 22. These inter-events steps may be attributed to selected configurations (a to c) as above. Theoretically three interruptions of blockages are expected for 4 modifications. Arrows indicate OsBp moiety and have direction; blocks indicate partial coverage of adjacent bases due to the presence of OsBp. The planar structure of OsBp prohibits full coverage of adjacent bases (not shown in the 2D configuration in FIG. 33 top). Note that there is more than one plausible configuration to rationalize type b and type c events.

FIG. 23: a) Histograms are shown for the fractional current blockade (DI/Iₒ), as well as the dwell times for 80mer ssDNA (ODN4 or L1 in Table 1; (SEQ ID NO:5) osmylated at R1 and R2 levels with Protocol A or B, respectively. In these experiments a solid-state SiN nanopore (1.6 nm wide and 3 nm long) with an applied bias voltage of 300 mV are used. Experiment was conducted with chambers from either side of the nanopore filled with a buffered solution containing 0.4M KCl, 4.8M urea, 1 mM EDTA, and 10 mM Tris, at pH 8.0. (T+C)-osmylated molecules (R2) show markedly greater dwell times as compared to unreacted (ODN4), and T-osmylated molecules (R1). The shaded region encapsulates all of the lower blockade peaks of the double Gaussian fits; stars indicate the locations of the higher blockade peaks. b) Concatenated events are shown for each molecule. Data is shown after low pass filtering at 100 kHz.

### DETAILED DESCRIPTION OF THE INVENTION

The present invention claims that nucleic acids may be osmylated independent of sequence, length, and composition using the same protocols for every nucleic acid including ssDNA, and dsDNA after denaturation. Extent of labeling is predictable and can be confirmed by a UV-vis assay described here by the inventor. The presence of the osmylated pyrimidine slows down translocation via suitable nanopores, both natural and solid-state, and exhibits discrimination between intact and labeled bases. Different electrophoretic properties, and hence discrimination, is also exhibited among the labeled pyrimidines themselves. Hence osmylated nucleic acids enable unassisted, nanopore-based sequencing with no limit in the length of the polynucleotide due to its enzyme-free implementation.

### Osmylation of T:

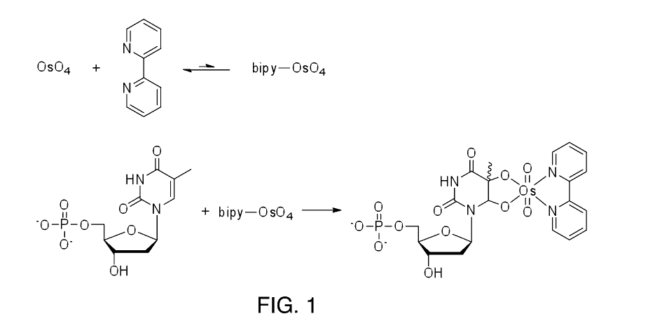

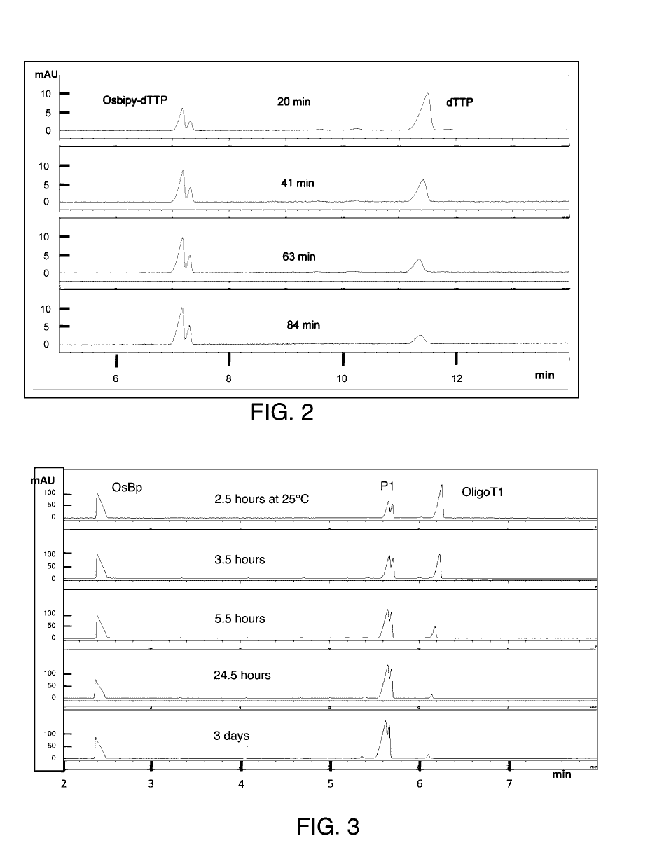

Earlier publications of others used Osmium tetroxide and amines at various experimental conditions to label pyrimidines. For a review see reference (Palecek, 1992). In one embodiment the present inventors prepared a 1:1 molar mixture of Osmium tetroxide (4% aqueous solution purchased from Electron Microscopy Sciences) and 2,2′-bipyridine (99+ purity purchased from Acros Organics) in glass vials in water at a final concentration of 15.75 mM each (stock solution of Osbipy or OsBp, see FIG. 1). Oligos (deoxy unless otherwise specified) were selected to be short and of specific sequence (see Table 1), so that they could be analyzed by capillary electrophoresis (CE) or High performance Liquid chromatography (HPLC) and provide full resolution of the products resulting from reaction with OsBp. It should be mentioned that OSO₄ is volatile and dangerous. Safety precautions must be taken when preparing the stocksolution and the reaction mixtures. Because the equilibrium constant is relatively small, most of the OSO₄ in the OsBp solution is also in free form, so the OsBp solutions and mixtures with oligos are equally dangerous.

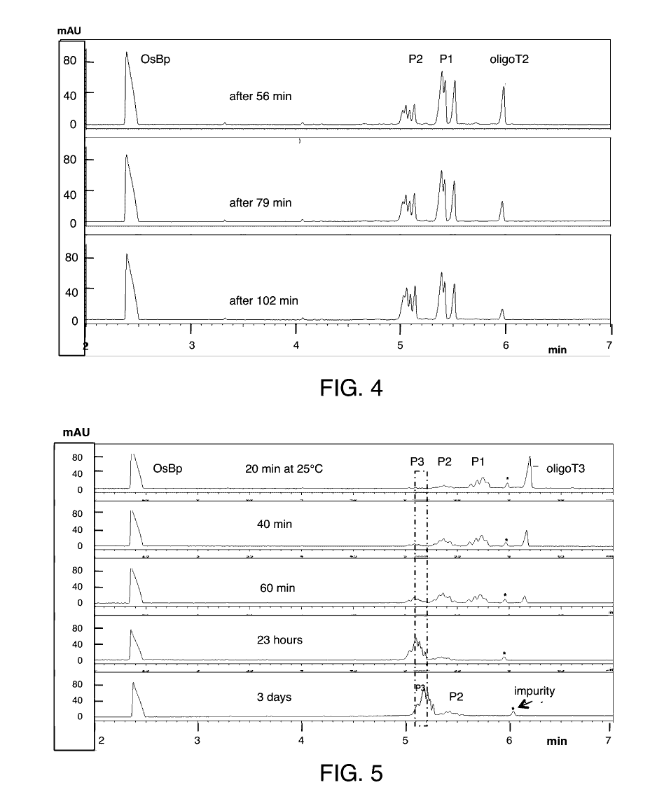

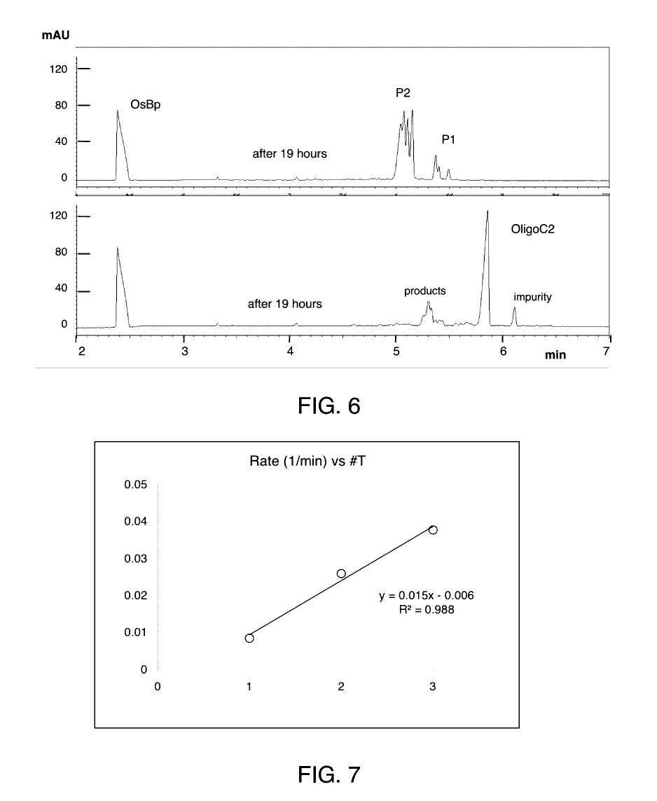

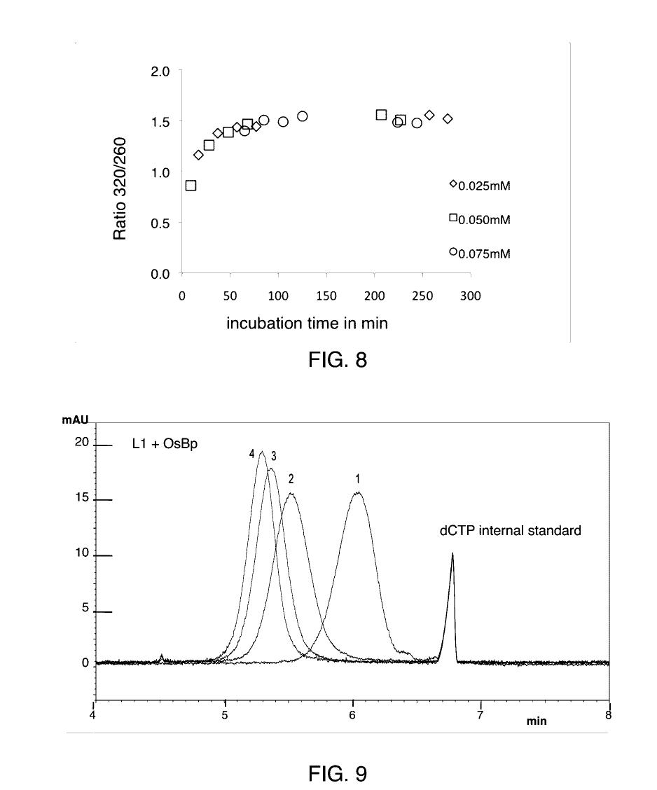

The 1:1 preparation of OsBp at a 15.75 mM was mixed with the selected oligos in water at different initial concentrations at room temperature and allowed to react, while it was monitored by CE (see FIGS. 3-7). The reaction was always conducted in a glass vial, in water, with no buffer added. Most buffers react with OsBp and lower its concentration and pH control was found to be unnecessary. Reaction mixtures need to be placed in glass vials, because other materials react with OsBp and lower its effective concentration yielding irreproducible results. Oligo disappearance and product formation were monitored automatically by CE over time (FIGS. 3-7). Our investigations yielded conditions at which full conversion to labeled products was evidenced analytically. Specifically, it is critically important with the 1:1 OsBp preparation that the concentration of the label in the reaction mixture with the oligo is at least 3 mM OsBp; lower concentrations do NOT yield full osmylation, even under prolonged incubation conditions. Also critically important is that the concentration of the base (in the oligo) to be osmylated is in the range of 0.10 to 0.15 mM, or 20 to 30-times lower than the OsBp concentration. However the actual concentration of the oligo does not influence the rate of reaction (as it should be under pseudo-first order conditions). This is evidenced in FIG. 8, where a factor of 3, from 0.025 to 0.075, makes no difference in the rate of product formation.

The present inventor also determined the selectivity of OsBp for T over C under the reaction conditions (water and room temperature) in more than one ways and Table 2 shows some of the results to indicate an initial selectivity of T:C=25±2. It should be noted that as the reaction of an oligo progresses and more of the T is labeled, the actual observed selectivity, i.e. the ratio of T(OsBp)/C(OsBp) decreases. Because the conditions recommended by this inventor are pseudo-first order conditions, percent pyrimidine osmylation can be predicted from the rates of the two processes, T-osmylation and C-osmylation (see more later). Table 3 provides specific examples that have all been validated experimentally. Hence the recommendation is to prepare a mixture of 3 mM OsBp and polynucleotide at, at least, a 20-fold lower concentration expressed in T equivalents, and incubate for 60 min. These conditions, Protocol A, will give 90% T(OsBp) and 6.5% C(OsBp) in any oligo (intrapolated from Table 3); other incubation times can be selected depending on the desired outcome.

In contrast to a published report from Chang, Beer, and Marzilli (1977, see page 37, 1^{st} paragraph) who were unable to find conditions to selectively osmylate T over C, the current inventor discovered such conditions and discloses them in this invention.

In contrast to published results from Nomura and Okamoto (2008), the present invention recommends conditions that lead to comparable reactivity of Ts independent of composition. The comparable reactivity is important because it leads to one protocol for T-osmylation for any nucleic acid. In one embodiment, illustrated in FIG. 7, the reactivity of T osmylation remains the same as a function of the number of Ts in an oligo, as seen by the proportionality of the rate of oligo disappearance with number of Ts in the oligo. If reactivity varied with number of Ts, then the line would curve up (increased reactivity), or down (decreased reactivity). It is likely that the harsch conditions used by Nomura and Okamoto (incubate with 100 mM of potassium osmate and 100 mM of potassium hexacyanoferrate, and treated the samples with piperidine at 90° C. for 20 min in order to cleave the phosphodiester bond at the oxidized T sites), resulted in the apparent difference of osmylation between isolated and tandem Ts.

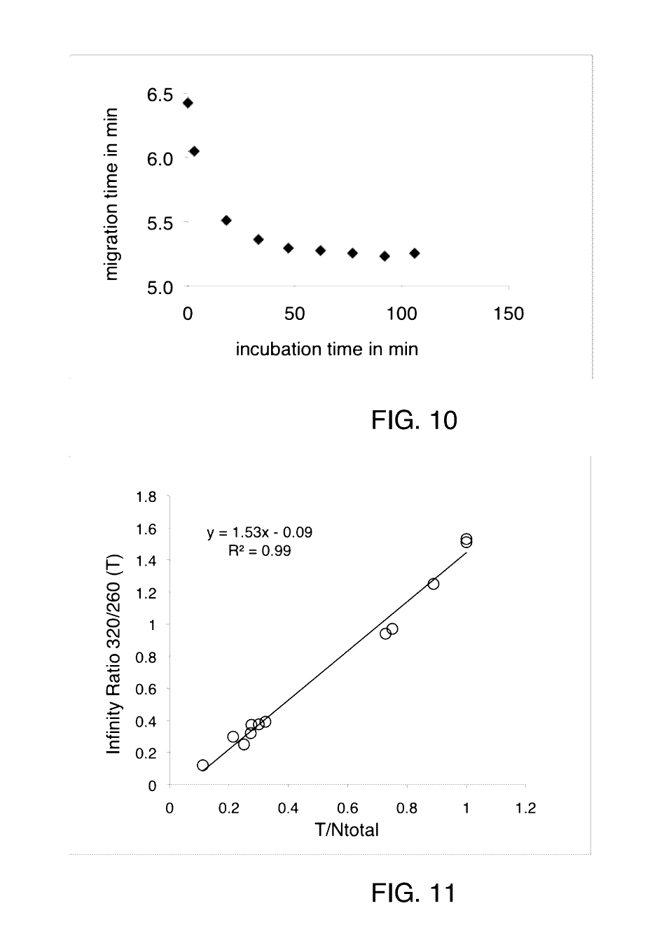

The present invention includes two different measures (or assays) for determining rate of final product formation (complete osmylation), in cases where the oligo is relatively long and resolution of the products, intermediate and final, is not feasible by analytical instrumentation, be that CE or HPLC. One is a UV-Vis assay and it will be described in detail below, and the other is monitoring the migration time (mt) by CE of the reacting oligo peak with incubation time. One should be reminded that by CE, OsBp migrates first, and the intact oligo migrates last. Osmylated oligo migrates between the two and the migration time (mt) is earlier with more osmylation. Once an oligo is above 10 to 15 nt long, then there is no good resolution, i.e. separate peaks for different products, but there is one “peak” that shifts to earlier times as a function of incubation and osmylation progress. Once the reaction is complete, the mt remains unchanged. FIG. 9 shows the peak of an 80-mer oligo (L1, Table 1; SEQ ID NO:5) that shifts to earlier mt with incubation time, while an internal standard (dCTP) does not move at all. Using dCTP as internal standard was possible due to the dramatic difference in reactivity between T and C. The observed mt with incubation time (t) are plotted in FIG. 10 and illustrate an exponential curve, as expected for a pseudo-first order reaction, which provides the rate of product formation (k_{obsd}) as the slope (absolute value) of a plot of LN (mt at time t-infinity mt) as a function of t, where LN is the natural log (see Kanavarioti et al. 2012). Please note that the intact oligo's mt is 6.5 min.

Rate determination of a process provides detailed mechanistic insights into a reaction and allows for predictability. This is a well-known concept, but its implementation is not simple. With short oligos, where analytical tools allow for each product to be monitored, we measured the rate of oligo disappearance, and the rate of final product formation by monitoring the oligo or the final product, respectively, as a function of incubation time. With the longer oligos disappearance of oligo is almost instantaneous due to statistical reasons. FIG. 7 estimates the rate of disappearance of an oligo that has 4 Ts or more by extrapolation; this rate is too fast for our instruments. However the rate of final product formation is slow and I measured it with the longer oligos either by following the migration time, or by following the absorbance at a wavelength that the intact oligo does not absorb. The following paragraph describes the UV-Vis assay that I invented; it is an assay that makes the determination of the rate of osmylation feasible for any polynucleotide, and as it will be shown later, correlates with the fraction or normalized number or osmylated Ts in an oligo.

### A Simple UV-Vis Assay to Determine Extent of Osmylation:

While investigating these reactions we made the observation, which confirmed earlier literature, that the osmylated product exhibits absorbance in the range of 300 to 340 nm, with a maximum around 310 to 320 nm. It is well known that intact oligos do not have any considerable absorbance in this range, so at the onset of the reaction the “oligo” peak does not show up at 320 nm, but as soon as product is forming the absorbance at 320 nm increases, in an exponential form due to the pseudo-first order conditions, and levels off once the reaction is complete. In order to minimize the effect of instrument sampling and other experimental variations, the absorbance was normalized by taking the ratio of R=320/260; for an example, see FIGS. 8 and 11. The Ratio obtained after completion of the osmylation process, called Infinity Ratio 320/260 (T) is reported in the 7^{th} column of Table 1, and plotted in FIG. 11 as a function of the fraction of # of Ts over the total number of nucleotides in the oligo. Please note that these oligos were the ones where final product formation was confirmed directly by CE, so this set of oligos was exploited as a “training set” to evaluate the existence of a correlation. The observation with this set of oligos of a linear relationship that goes via the intercept (0,0) clearly suggests that every single dT(OsBp) is a chromophore that equally contributes to the total absorbance of the product. This conclusion is confirmed by comparing the practically identical values of Infinity Ratio 320/260 (T) R=1.53 and R=1.51, respectively for dTTP and oligoT8 (Table 1).

As it will be shown later, we were able to confirm that osmylation of C, even though a much slower reaction follows the same principles as T-osmylation, and hence the UV-Vis assay can be used for both pyrimidines (more on this later). All the initial investigations were conducted using analytical tools, such as CE or HPLC, that allow for resolution of a mixture of starting material and products. However, once purified from the excess OsBp, the solution of the pure osmylation product can be measured by any UV-Vis spectrophotometer and provide the value R 320/260. The actual concentration of the labeled polymer does not need to be known, but can be determined from the Absorbance at 260 nm because osmylated oligo and intact oligo have comparable extinction coefficient at 260 nm. Purification methods to remove small molecules from polymers are many (look up nucleic acid purification kits) and we validated one of them, namely the spin columns TC FC-100 from TrimGen. One or two passes are sufficient to remove up to 12 mM of OsBp, with excellent recovery of the labeled polymer.

The independence of T and/or C-osmylation on composition, sequence, and length could have not been predicted a priori. Actually the exact opposite is more in tune with scientific intuition. I only became aware of it after listing the determined rates for product formation (see 4^{th} column in Table 1) for a variety of oligos. All the rates are practically the same with k_{obsd}=0.042±0.003 per min under the experimental conditions (in water, room temperature and 3 mM OsBp (1:1 preparation). Evidence for comparable rates imply that the same protocol predictably osmylates every oligo, and the % T and % C osmylated given in Table 3 are valid for any oligo. Later it was shown that this conclusion is valid for a 7459 nt long circular DNA (ssM13mp18) (see provisional patent, Kanavarioti, 2015), and it is only then that T-osmylated nucleic acids exhibit specific and substantial utility for sequencing purposes.

### C-Osmylation:

When we published the data on T-osmylation the recommended conditions for C-osmylation were 50 h at 35° C. in the presence of 11.6 mM OsBp (Kanavarioti et al., 2012). However we had no evidence whether or not C-osmylation is independent on composition, length, and sequence, and we also couldn't confirm extent of labeling because R 320/260 for dC(OsBp) was R≈1.0. Hence we set up to study C-osmylation in detail and Tables 4 and 5 list the oligos/DNA used and the results obtained. First the assay was optimized so that both dT(OsBp) and dC(OsBp) could be satisfactorily monitored, and the new “best mode” R is 312/272, reported in the two last columns of Table 5. R1 (312/272) refers to Protocol A to practically osmylate Ts, and R2 (312/272) refers to Protocol B to practically osmylate both T+C. Protocol A (1^{st} optimization) recommends the use of 50 to 200 ng/uL DNA with 3.15 mM OsBp in water in stoppered glass vial, 60 min incubation at room temperature and purified within couple of minutes with TrimGen. After Protocol A, 90% of T is osmylated and 6.5% of C is osmylated. Protocol B (1^{st} optimization) recommends use of 50 to 200 ng/uL DNA with 14.2 mM OsBp in stoppered glass vial, 11 hours incubation at room temperature, followed by TrimGen purification; Protocol B results in 100% (T+C)(OsBp). Notably other purification methods may work equally well, but need to be validated.

FIG. 12 illustrates an example for an oligo with 6Cs that happened to be fully resolved by CE. Monitoring this reaction to completion (not shown here) is one of the ways we evidenced complete C-osmylation. FIG. 13 illustrates the two separate products obtained from circular ssM13mp18 (7459 nt long, M13 in figure) following the same protocols (A and B, previous paragraph) as for short oligos. With M13 we experimented with 6 different osmylation conditions including the presence of urea that is known to denature secondary structure and it was surprising to this inventor that no urea was necessary with M13. Apparently OsBp concentrations at 10 mM or higher have denaturing properties. Convincing evidence for the predictability of the labeling with OsBp is presented in FIG. 14 (T) and FIG. 15 (T+C). The data in these figures are all the ones reported in Table 5 and include the M13 data for both T- and T+C-osmylation.

### Quality Control Assay:

Based on FIG. 14, one can calculate the theoretically expected value R1(312/272) of a known oligo/DNA following osmylation by Protocol A from R1=2.21×T/Nₜₒₜₐₗ. Based on FIG. 15 one can calculate the theoretically expected value R2(312/272) following osmylation by Protocol B from R2=2.01×(T+C)/Nₜₒₜₐₗ. In all the oligos/DNA we have osmylate so far, around 70, the assay always worked. This is why we claim that this assay R(312/272) can be used as a quality control assay (±3%) to confirm that protocols A and B have worked as expected. Evidently, one can use the assay to determine extent of osmylation, even if one does not use the recommended protocols, because this assay is based on the thermodynamic property of the osmylated polymer. Please note that the “best mode Protocols A and B”, described below, were designed in such a way that with respect to the assay are practically equivalent with the Protocols A and B (1^{st} optimization).

### Stability of Osmylated Polymers:

Prolonged incubation of the osmylated polymers over days at room temperature and in the presence of OsBp as high as 14 mM, show no detectable changes as evidenced by CE. In addition, OsBp exhibits no reactivity towards the purines and no detectable propensity towards degradation of the backbone or any other bond in the polymer, as evidenced by accounting for every peak in the CE profiles. However dC(OsBp) hydrolyzes to form dU(OsBp) with about 1 to 2% per hour, and this observation prompted this inventor to optimize conditions, so that osmylation of C is expedited, and dC(OsBp) transformation to dU(OsBp) becomes minimal.

### Best Mode Osmylation Protocols:

In order to suppress the transformation of dC(OsBp) to dU(OsBp) which we evaluated as 1 to 2% per hour under the typical C-osmylation conditions, we prepared a novel OsBp formulation/stock solution. OsBp new preparation is still 15.75 mM in OSO₄, but prepared in saturated 2,2′-bipyridine using a 5 to 10-fold molar excess of the later. After vigorous mixing of the two components, the supernatant is removed and used as the new stock solution (OsBp 15.75 mM in saturated 2,2′-bipyridine). Saturated 2,2′-bipyridine in water is approximately 30 mM as indicated in the literature. Experiments and kinetic determinations with the new stock solution revealed that the reactivity is much higher about a 4-fold compared to the OsBp 1:1 preparation. Hence we recommend “best mode” Protocol A as 60 min incubation in 1.575 mM OsBp (sat. bipy), and “best mode” Protocol B as 110 min incubation in 12.6 mM OsBp (sat. bipy). Please note that the stock solution is saturated in bipyridine, because of the way it was prepared. However the resulting reaction mixtures, because they are accordingly diluted (either to 1.575 mM or to 12.6 mM) are no longer saturated in bipyridine. Based on the new reactivities, which will be published shortly including documentation, Protocol A results to 95% T-osmylation and 8% C-osmylation; Protocol B results to over 99.99% T-osmylation and 99.99% C-osmylation.

Osmylation of Ribooligonucleotides and ssRNA:

As mentioned osmylation is a reaction with the C5-C6 double bond of the pyrimidines, and it is not influenced by the presence of the sugar or the phosphate tail. Hence it is anticipated that oligoribonucleotides bases rA, rG, rU, and rC will react with the same reactivity as their deoxy-counterparts. The order of OsBp reactivity for the nucleotides is: dT>5′Me-dC>dU>5′MeOH-dC>dC, with U being only 2 to 3 times more reactive compared to C. Hence to osmylate a ribooligonucleotide comprising of U and C, we recommend to follow best mode Protocol B above.

### Nanopores as Sequencing Devices:

As discussed in the “Background” nanopores have been pursued as single molecule detection devices, and the corresponding progress in manufacturing, parallelization, and commercialization of such platforms have made them very promising tools for nucleic acid sequencing. However years of experimentation has also unraveled their shortcomings. One major issue is the chemical comparability of the nucleobases and the associated inability of a nanopore to discriminate them clearly. The realization that OsBp adds a four-fold mass on the reacting pyrimidine (FIG. 1) and the fact that our conditions promote homogeneous and predictable osmylation for any nucleic acid, led the present inventor to propose the use of labeled nucleic acids as surrogates for nanopore-based sequencing (see Publication 2). Recent experiments at our collaborators (Publications 3 and 5) using the osmylated oligos prepared by this inventor yielded promising results, as detailed below.

Under the influence of voltage osmylated oligos traverse suitable nanopores, both natural and man-made. Translocation is slow and the current is obstructed. The nanopore clearly senses the presence/absence of OsBp, and in the case of α-HL there is clear discrimination of the osmylated pyrimidine based on the bases' identity. These observations (see Table 7) provide proof-of-principle for nanopore-based sequencing.

In some embodiments translocation via a-Hemolysin nanopore (α-HL) was evaluated. FIG. 17 shows a representation of α-HL and the translocating nucleic acid. Table 6 lists oligos, their sequences, SEQ ID NO, and purity, tested with α-HL and Table 7 lists the observed electrophoretic parameters of these oligos. The extent of osmylation R1 or R2 is also included in the Table indicating Protocol A or Protocol B treatments (1^{st} optimization), respectively. It is seen that tested osmylated oligos obstruct the current more compared to the control oligo, dA₂₀ (SEQ ID NO:28). Under the conditions of the experiments listed in Table 7, only 14% of the typical current (Iₒ) remains upon dA₂₀ translocation, whereas translocation of the osmylated oligos yields current that is in the range of 3 to 12% of Iₒ. The more extensive the osmylation, the more current is obstructed. Still the effect on the current obstruction is small compared to the effect of osmylation on the translocation speed or dwell time.

As seen in FIG. 22 (middle) within the translocation event of an osmylated oligo, there are “spikes” (of more current obstruction) attributed to the passing of each osmylated base or unit. Hence oligo dA₂₅-pGEX3′ R1 with 4 T(OsBp) should have shown 4 spikes (FIG. 22, middle). However this figure shows that this oligo exhibits translocation events with one solid spike, two or three, but not four. FIG. 22 (top) is a representation of an osmylated DNA to illustrate that due to the size of the OsBp moiety and its directionality with respect to the strand, there is substantial overlap between OsBp moieties, even when the osmylated bases are one base apart from each other. FIG. 22 (bottom) is a representation that illustrates this overlap and rationalizes the observation of a continuous, two, or three spikes.

Sequencing Strategy: FIG. 21 illustrates the basic strategy for sequencing dsDNA or any ds polynucleotide using only OsBp or an equivalent X-substituted OsBp. This strategy is enabled by the nanopore sensing of intact base, dT(OsBp), and dC(OsBp) labeled as 0, 1, and 2, respectively in FIG. 21. This strategy would be easy to understand and implement, if OsBp did not extend to the neighboring bases. For example, Protocol A osmylation that yields primarily Ts, would identify Ts via nanopores. Then nanopore based sequencing of target strand by Protocol B would identify both T+C. Using the complementary strand after Protocol A and B osmylation and nanopore-based sequencing would provide the positions of A and G, that correspond to the T and C of the target strand, and sequencing of the target strand would be accomplished.

Because of the evidence that OsBp extends over the neighboring base, we now recommend instead of Protocol A, osmylation to about 5%, and instead of Protocol B, osmylation with Protocol A for both strands. This revised strategy will avoid complicating the analysis of overlapping OsBp moieties. There are again four labeled polymers to be sequenced, but the levels of osmylation are different. Because of the homogeneity of the labeling process the solution that contains the 5% osmylated target strand will contain many strands, where not all Ts are osmylated, but in the mixture every T will appear osmylated in some of the strands, due to the homogeneous non-biased labeling. Nanopore-based sequencing using dwell time as the critical parameter will identify all the Ts. Furthermore the few dT(OsBp) per strand will be used as markers, so that the number of intact bases between two markers can be determined. This is because, as shown in experiments of other investigators, translocation time is proportional to the number of bases when the bases are intact. All the translocation events will be compared and aligned to provide a consensus strand that incorporates all the Ts, as well as all the intact bases between them. Sequencing the solution with the Protocol A osmylation (ii) will provide all the dC(OsBp) positions, in analogy to the dT(OsBp) methodology described above. Again due to the homogeneity of C-osmylation, each strand will have a small number of dC(OsBp), (8% with the best mode Protocol A), and many Cs intact. However among all the osmylated polymers in the solution each C will appear osmylated in some strand(s). So with Protocol A, identification of Cs is accomplished in addition to confirmation of Ts and intact purines in between. Since the dwell time for dC(OsBp) is about 0.36 ms at 120 mV whereas the dwell time for dT(OsBp) is about 0.15 ms at 120 mV, “spikes” due to C passing will be about 2-times slower compared to spikes due to T passing, and discrimination will be clear. For a more detailed description of this approach please see Publication 4.

### Identification of Non-Canonical Bases Including 5′Me-dC and 5′OHMe-dC:

Current interest includes, in addition to the genome, sequencing the transcriptome and the epigenome. We already discussed an approach for pyrimidine sequencing within ssRNA. Osmylation will also denature ssRNAs and tRNAs that consist of several double-stranded regions. Denaturation upon osmylation is expected based on the observation that circular ssM13mp18 became osmylated using the same protocols A or B, just like the short oligos (see FIGS. 14 and 15). Distinguishing the different forms of methylated C by nanopore-based sequencing of osmylated DNA is another application of the invented technology. The selectivity of OsBp for T over C is high, the selectivity for 5-MeC lies in between, and the selectivity for 5-OHMeC is 2-fold higher compared to C. OsBp selectivity for the different methylated Cs will determine their relative distribution after Protocol A osmylation. Discrimination based on residual current as well as dwell time will be additional parameters to facilitate identification. Determination of cytosine methylation levels, i.e. epigenome, is concomitant with the basic sequencing described above, and will not require additional analysis and time commitment.

In conclusion, these data demonstrate that osmylated nucleic acids can be prepared easily, and accurately characterized. They have specific and substantial utility in nanopore-based sequencing applications with projected more accurate, less expensive, much faster, and less ambiguous features compared to the current state of the art in DNA sequencing. While embodiments have been illustrated and described, it will be appreciated that various changes can be made therein without departing from the spirit and scope of the invention.

What is claimed is:

1. Methods for preparing osmylated nucleic acids (osmylated or labeled polymers) comprising:
Using Osmium tetroxide 2,2′-bipyridine of a recommended preparation at recommended conditions in order to selectively label T or T+C or at alternative levels of osmylation;
purifying the product by one or more purification methods to remove the unreacted label;
and using one or more analytical methods to characterize the article and confirm extent of labeling by the disclosed assay.

2. A method of determining the sequence of pyrimidines of the osmylated polymer, comprising:
applying an electric field across a nanopore disposed between a first conductive liquid medium and a second conductive liquid medium and
measuring an ion current to provide a threshold amount in the absence of the article and then
measuring the changed current pattern (i-t) while the labeled polymer traverses through the nanopore.

3. A method of assigning changes in i-t measurements from the threshold amount to a T-osmylated or a C-osmylated unit, based on comparison to i-t patterns with labeled polymers of known sequence; and hence inferring the pyrimidine units of the sequence of the target nucleic acid. Repeating this procedure for the complementary strand in order to assess the position of the pyrimidines that correspond to the missing purines of the target strand.

4. The method of claim 1, wherein the label is Osmium tetroxide 2,2′-bipyridine(X-substituted).

5. A kit for performing the method of claim 1, comprising, in separate compartments,
a) the label,
b) the purification component,
c) instructions for using a) and b) in series, and
d) instructions to do quality control test after performing b).

6. A kit for performing the method of claim 4, comprising, in separate compartments,
a) the label,
b) the purification component,
c) instructions for using a) and b) in series, and
d) instructions to do quality control test after performing b).


SEQUENCE LISTING (34 sequences)

SEQ ID NO: 1 - DNA, 11 bp, artificial sequence
  synthetic
aataataataa

SEQ ID NO: 2 - DNA, 11 bp, artificial sequence
  synthetic
ttattattatt

SEQ ID NO: 3 - DNA, 11 bp, artificial sequence
  synthetic
aacaacaacaa

SEQ ID NO: 4 - DNA, 11 bp, artificial sequence
  synthetic
ttcttcttctt

SEQ ID NO: 5 - DNA, 80 bp, artificial sequence
  synthetic
ctcagagttccaaggtgagtgagtgagtggtccttccttccttccttccggtgagtgagtgagtggacggtaagccattt

SEQ ID NO: 6 - DNA, 90 bp, artificial sequence
  synthetic
ctcagagttccaaggtgagtgagtgagtggtccttccttctccttccttccttccttccggtgagtgagtgagtggacggtaagccattt

SEQ ID NO: 7 - DNA, 80 bp, artificial sequence
  synthetic
ctcagagttccaaggagagagagttagtggtccttccttccttccttccggtgattgagagagaggacggtaagccattt

SEQ ID NO: 8 - DNA, 89 bp, artificial sequence
  synthetic
ctcagagttccaaggcactcacgcactcacggacttcaggacttcaggacttcaggacttcaggacttcaggacgacggtaagccattt

SEQ ID NO: 9 - DNA, 20 bp, artificial sequence
  synthetic
cgcggggtggagcagcctgg

SEQ ID NO: 10 - DNA, 57 bp, artificial sequence
  synthetic
tagctcgtcgggctcataacccgaagatcgtcggttcaaatccggcccccgcaacca

SEQ ID NO: 11 - DNA, 20 bp, artificial sequence
  synthetic
ccaggctgctccaccccgcg

SEQ ID NO: 12 - DNA, 57 bp, artificial sequence
  synthetic
tggttgcgggggccggatttgaaccgacgatcttcgggttatgagcccgacgagcta

SEQ ID NO: 13 - DNA, 20 bp, artificial sequence
  synthetic
atactggtggagcagtttgg

SEQ ID NO: 14 - DNA, 20 bp, artificial sequence
  synthetic
cgcgaagtggagcagnctgg

SEQ ID NO: 15 - DNA, 20 bp, artificial sequence
  synthetic
cgcgaagtggagcagcctgg

SEQ ID NO: 16 - DNA, 16 bp, artificial sequence
  synthetic
aaaacccaacccaaaa

SEQ ID NO: 17 - DNA, 16 bp, artificial sequence
  synthetic
aaaaaccccccaaaaa

SEQ ID NO: 18 - DNA, 16 bp, artificial sequence
  synthetic
aaacacacacacacaa

SEQ ID NO: 19 - DNA, 15 bp, artificial sequence
  synthetic
ttttttttttttttt

SEQ ID NO: 20 - DNA, 20 bp, artificial sequence
  PCR primer
ggccacgcgtcgactagtac

SEQ ID NO: 21 - DNA, 23 bp, artificial sequence
  synthetic
ccgggagctgcatgtgtcagagg

SEQ ID NO: 22 - DNA, 23 bp, artificial sequence
  synthetic
gggctggcaagccacgtttggtg

SEQ ID NO: 23 - DNA, 20 bp, artificial sequence
  synthetic
cgctctagaactagtggatc

SEQ ID NO: 24 - DNA, 20 bp, artificial sequence
  synthetic
cttgggtggagaggctattc

SEQ ID NO: 25 - DNA, 20 bp, artificial sequence
  synthetic
aggtgagatgacaggagatc

SEQ ID NO: 26 - DNA, 20 bp, artificial sequence
  synthetic
agagtttgatcctggctcag

SEQ ID NO: 27 - DNA, 23 bp, artificial sequence
  synthetic
agcggataacaatttcacacagg

SEQ ID NO: 28 - DNA, 20 bp, artificial sequence
  synthetic
aaaaaaaaaaaaaaaaaaaa

SEQ ID NO: 29 - DNA, 20 bp, artificial sequence
  synthetic
aaaaaaaaaataaaaaaaaa

SEQ ID NO: 30 - DNA, 20 bp, artificial sequence
  synthetic
aaaaaaaaaacaaaaaaaaa

SEQ ID NO: 31 - DNA, 20 bp, artificial sequence
  synthetic
aaaaaaaaaanaaaaaaaaa

SEQ ID NO: 32 - DNA, 20 bp, artificial sequence
  synthetic
aaaaaaaaaanaaaaaaaaa

SEQ ID NO: 33 - DNA, 48 bp, artificial sequence
  synthetic
aaaaaaaaaaaaaaaaaaaaaaaaaccgggagctgcatgtgtcagagg

SEQ ID NO: 34 - DNA, 48 bp, artificial sequence
  synthetic
ccgggagctgcatgtgtcagaggaaaaaaaaaaaaaaaaaaaaaaaaa